(19) United States
(12) Patent Application Publication
(10) Pub. No.: US 2021/0386083 A1
(43) Pub. Date: Dec. 16, 2021

(54) METHOD FOR PRODUCING A PHOSPHOLIPID CONCENTRATE FROM A DAIRY COMPOSITION

(71) Applicant: EUROSERUM, Port-sur-Saône (FR)

(72) Inventors: Charlotte Bastian, Noidans-lès-Vesoul (FR); Lydie Laurens, Héricourt (FR); David Girardot, Montenois (FR)

(73) Assignee: EUROSERUM, Port-sur-Saône (FR)

(21) Appl. No.: 17/282,704

(22) PCT Filed: Oct. 3, 2019
(86) PCT No.: PCT/FR2019/052344

§ 371 Date: Apr. 2, 2021

(30) Foreign Application Priority Data
Oct. 3, 2018 (FR) FR1859157

(51) Int. Cl. A23C 9/146; A23C 9/142; A23C 17/00; B01D 61/14; B01D 61/16; B01D 71/02; B01D 69/02; B01J 39/07; B01J 39/20; B01J 47/02

(52) U.S. Cl. CPC A23C 9/146; A23C 9/142; A23C 17/00; B01D 61/147; B01D 61/16; B01D 71/02; B01D 2325/02; B01J 39/07; B01J 39/20; B01J 47/02; B01D 2311/04; B01D 2311/2623; B01D 2315/16; B01D 69/02

(57) ABSTRACT

The method for producing a phospholipid concentrate derived from a liquid dairy composition comprising at least caseins and phospholipids, includes at least the steps of: —passing the dairy composition into an ion-exchange column on a cationic resin, —concentrating the phospholipids of the calcium-depleted dairy composition by means of controlled transmembrane pressure gradient microfiltration, and —recovering the retentate from the microfiltration and obtaining a phospholipid concentrate.

### FIELD OF THE INVENTION

The general field of the invention is that of methods for concentrating phospholipids which are present in a dairy product, and more particularly for concentrating phospholipids from buttermilk.

### PRIOR ART

Phospholipids are important constituents within cell membranes. They are found in the vital organs such as the brain, the liver, and the kidneys. They intervene in many biological mechanisms such as the regulation of inflammatory reactions, the regulation of the immune system (combating infections in infants, especially preterms), combating cancer of the colon, intestinal maturation in the fetus, lowering cholesterolemia, preventing cardiovascular diseases (antiatherogenic function), reducing allergies in young children, helping to detoxify the liver, enhancing cognitive development (particularly in infants), combating Alzheimer's disease, or else developing the memory.

Phospholipids are naturally present in maternal milk. In industry, they are therefore found in various products and coproducts that are obtained from the milk of bovine origin or of other animal origin. Dairy phospholipids are contained in particular in a fine membrane of biological origin called MFGM (Milk Fat Globule Membrane). The MFGM is a bilayer formed in the mammary gland, which surrounds the fat globules and stabilizes the emulsion of the butyric fatty matter in the aqueous phase.

MFGM is a complex mixture of proteins and lipids. The lipid fraction of the MFGM obtained from milk of bovine origin is composed of a mixture of 65% of neutral lipids (for example, tri- and diglycerides, cholesterol) and 35% of polar or complex lipids (including 26% of phospholipids and 9% of sphingolipids). The class of the phospholipids breaks down as follows: 30% phosphatidylethanolamine (PE), 35% phosphatidylcholine (PC), 5% phosphatidylinositol (PI), 3% phosphatidylserine (PS), and 22% sphingomyelin (SM) (Danthine et al., 2000).

Dairy phospholipids (of animal origin) are the closest phospholipids in qualitative terms to maternal phospholipids, compared in particular with phospholipids of plant origin (soy in particular), which do not contain sphingomyelin.

Dairy phospholipids are found in particular in the buttermilk, called milk buttermilk, that constitutes the aqueous phase resulting from the rupture of the MFGM during churning of the cream, and which is recovered during the formation of grains of butter. On average, the phospholipid content of the buttermilk represents 20% of its total lipids. This amount varies depending on the origin of the cream, the type of process used, but also according to the stage of lactation and the race of the cow. Phospholipids are also found in the secondary buttermilk, buttermilk melt or concentrated buttermilk (also called butter serum), obtained by concentrating butter, and in the beta-serum obtained directly from the cream.

On the industrial scale, phospholipids are generally obtained by processes of physical extraction (grinding, filtration, centrifugation, fractionation followed by precipitation) and solvent extraction (with acetone in general).

An interest is understood to exist in concentrating the phospholipids contained in buttermilk, particularly for exploiting the nutritional and emulsifying properties of the concentrate.

The publication Microfiltration of Butter Serum Upon Casein Micelle Destabilization, Rombaut et al, J. Dairy Sci. 89:1915-1925 describes an attempt to manufacture a phospholipid concentrate in a concentrated buttermilk. This manufacture induces a concentration by microfiltration at 50° C. on a cellulose acetate membrane with a pore diameter of 0.15 μm, under a transmembrane pressure of 1 bar. Prior to the microfiltration, attempts are made to dissociate the casein micelles present in the buttermilk. The reason is that the size of these micelles is comparable with that of the MFGM particles, thereby making it impossible to use microfiltration to purify the phospholipids from the buttermilk. To accomplish this, the dissociation is executed by adding sodium citrate or ethanol to the buttermilk. Although micelle dissociation is effective, especially by addition of sodium citrate, membrane fouling is observed and the resulting photolipid retention rate is inadequate.

### DRAWBACKS OF THE PRIOR ART

Besides an inadequate phospholipid retention rate, the method from the aforementioned publication involves the use of compounds such as sodium citrate and/or ethanol which are undesirable for use of the concentrate in the nutritional sector, and more particular as an ingredient of infant formula.

### OBJECTIVES OF THE INVENTION

The invention is directed primarily to a method for phospholipid concentration in a dairy composition by a membrane process, providing simultaneously a satisfactory phospholipid retention and a sufficient protein transmission, subject to the proviso that this method must be free of any compound which is undesirable and/or incompatible with the intended applications, particularly in the nutritional sector.

### SUMMARY OF THE INVENTION

To this end, the method of the invention for producing a concentrate of phospholipids from a liquid dairy composition comprising at least caseins and phospholipids is essentially characterized in that it comprises at least the steps of:
passing the dairy composition over cationic resin in an ion exchange column,
concentrating the phospholipids of the calcium-depleted dairy composition by controlled transmembrane pressure gradient microfiltration, and
recovering the microfiltration retentate and obtaining a phospholipid concentrate.

The method of the invention may also comprise the following optional features, considered in isolation or according to all of the possible technical combinations:
the dairy composition comprises milk buttermilk and/or concentrated buttermilk.
the ratio between the protein concentration factor and the phospholipid concentration factor is less than 1, each of said concentration factors corresponding to the ratio between the mass fraction of solids in the retentate and the mass fraction of solids in the buttermilk before microfiltration, and in that the protein retention rate induced by the microfiltration is less than 70%.
the ratio between the protein concentration factor and the phospholipid concentration factor is less than 0.9.
the cationic resin of the ion exchange column is a weak cationic resin.
a diafiltration is carried out during the phospholipid concentration step.
the diafiltration is applied for a volume concentration factor of less than 3.
the phospholipid concentration step is carried out at 50° C.
the tangential velocity applied to the microfiltration is greater than 4 m/s.
the mean pore size of the microfiltration membrane is less than 0.8 micrometer.
the mean pore size of the microfiltration membrane is less than 0.5 micrometer.
the transmembrane pressure is between 0.4 bar and 2 bar.
the transmembrane pressure is between 0.6 bar inclusive and 1 bar inclusive.
according to a first variant, the phospholipid concentration step is carried out on a permeability gradient membrane.
   the permeability gradient membrane has a mean pore size of 0.1 micrometer.
   the transmembrane pressure is maintained at 1 bar.
   a continuous diafiltration is applied when the volume concentration factor is between 1.2 and 3.
   the concentration step is carried out until the volume concentration factor of the dairy composition is between 2.5 and 5.
according to a second variant, the phospholipid concentration step is carried out on a ceramic membrane coupled to a system for harmonizing the transmembrane pressure.
   the mean pore size is between 0.1 and 0.2 micrometer inclusive.
   the transmembrane pressure is maintained at 0.6 bar.
   a continuous diafiltration is applied when the volume concentration factor is between 1.2 and 4.
   the concentration step is carried out until the volume concentration factor of the dairy composition is between 2.5 and 5.

### DESCRIPTION OF THE FIGURES

Further features and advantages of the invention will emerge clearly from the description thereof below, which is indicative and in no way limitative, with reference to the following figures:

FIG. 1 illustrates the protein retention rates as a function of the transmembrane pressure applied in a membrane concentration process coupled to a system for harmonizing the transmembrane pressure, preceded or not by a demineralization step,

FIG. 2 illustrates the amount of Total Nitrogenous Matter found in a centrifugation pellet at 31500 rpm, relative to the percentage of Initial Nitrogenous Matter in a concentrate obtained by the method of the invention, and a concentrate obtained without demineralization,

FIG. 3 illustrates the weight of water relative to the weight of Total Nitrogenous Matter in the same centrifugation pellet of a concentrate obtained by the method of the invention, and a concentrate obtained without demineralization,

FIG. 4 illustrates the particle size spectrum of a concentrate obtained by the method of the invention, and of a concentrate obtained without demineralization, in the presence of EDTA, and

FIG. 5 illustrates the particle size spectrum of a concentrate obtained by the method of the invention, and of a concentrate obtained without demineralization, in the presence of EDTA and SDS.

### DETAILED DESCRIPTION OF THE INVENTION

The method for producing a phospholipid concentrate of the invention from a dairy composition containing caseins and phospholipids comprises two main steps: the first is a step of dissociation of the caseins, and the second is a step of concentration by controlled transmembrane pressure gradient microfiltration.

The starting material used is milk buttermilk or concentrated buttermilk, owing to the high phospholipid content (approximately 20%) of its lipids. Use may alternatively be made of a mixture of milk buttermilk and concentrated buttermilk. The method of the invention is also applicable to any other type of buttermilk, and more generally to any dairy composition comprising caseins and phospholipids.

The casein which is present in dairy compositions and more particularly in buttermilk is in micelle form, and has a size similar to the fragments of MFGM (Milk Fat Globule Membrane) which contain the phospholipids, the other compounds present in buttermilk being different in size. It is therefore the case that if the buttermilk comprising casein in micelle form were to be passed through a membrane, both the proteins and the phospholipids would be retained, and this would not permit preferential concentration of the phospholipids.

The task is therefore to reduce the size of the casein micelles in order to decrease the protein retention rate on the membrane. To accomplish this, and as described in the aforementioned publication, it is known practice to chelate the calcium in the casein micelles by adding a dissociating agent such as sodium citrate or ethanol. However, besides the impossibility described in said publication of producing the intended concentrate, techniques of this kind using solvents give rise to a loss of the nutritional qualities of the resulting concentrate.

According to the invention, the size of the casein micelles is reduced by removing the calcium by ion exchange on a cationic resin. In this way, the casein micelles dissociate to form particles with much smaller sizes than the lipid fragments containing the phospholipids.

The cationic resin used in the demineralization column is a weak resin whose affinity for divalent ions is greater than for monovalent ions, thereby allowing the majority exchange of the calcium. The resin is regenerated in monovalent cation form, such as in potassium, sodium or hydrogen form, for example. Preference is given to regeneration in sodium and potassium form, since it avoids the need for the buttermilk to be acidified (and therefore for a base to be added at the column exit) and avoids aggregation of the caseins in the case of regeneration in hydrogen form. The buttermilk exiting the demineralization column therefore has free casein submicelles and a reduced calcium content. This buttermilk will be referred to as “calcium-depleted buttermilk or dairy composition in the remainder of the description”.

According to the invention, the calcium-depleted buttermilk at the column exit is subsequently subjected to a controlled transmembrane pressure gradient microfiltration, from which the retentate is recovered and forms the phospholipid concentrate. The application of a controlled transmembrane pressure gradient microfiltration is carried out in the context of the invention for the following reasons: in a membrane filtration, the separation performance is enhanced firstly by increasing the tangential velocity of the product along the membrane, and secondly by managing the transmembrane pressure. The effect of increasing the tangential velocity is a substantial loss of head between the entry and the exit of the product, and in this way a transmembrane pressure gradient is formed along the membrane. The tangential velocity therefore makes it difficult to manage the transmembrane pressure. Technologies have therefore been developed in order to overcome this problem. The first known technology is the UTP system, referring to the addition of a recirculation system for the permeate, so as to compensate the permeate-side pressure relative to the retentate-side pressure undergone. The second known technology is that of permeability gradient—PG—membranes, which have the feature of possessing a permeability gradient along the membrane at the level of the filtration support, so as to lower the transmembrane pressure gradient. Other membranes exist with the same system of permeability gradient, whether by structural modifications to the support or to the filtration layer. These membrane technologies are identified in the remainder of the description as “controlled transmembrane pressure gradient microfiltration” technologies.

Concentration may therefore be carried out on a permeability gradient (PG) membrane, sold for example under the name Membralox® GP, or on a ceramic membrane coupled to a system for harmonizing the transmembrane pressure, also referred to as a UTP (Uniform Transmembrane Pressure) system. Preference is given to using a pressure gradient membrane, because of the simplicity of the equipment. Other, equivalent technologies may be used, such as the tubular ceramic membranes sold under the name ISOFLUX™.

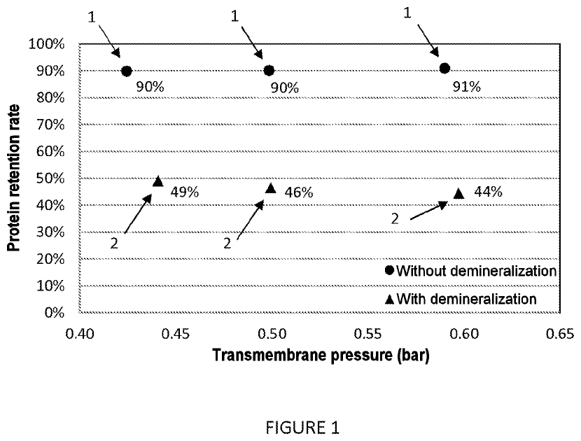

Reference is made to Tables 1a and 1b below, and also to FIG. 1, for a description of the tests carried out and an observation of the efficacy of the concentration of phospholipids by the method of the invention.

For the tests carried out with the UTP system, the buttermilk is fresh, or is prepared to 8% by mass of solids, from buttermilk powder. The tests take place as batch tests, with recirculation of the retentate and continuous withdrawal of the permeate. Samples of retentate and permeate are taken simultaneously. In order to ensure the best trade off between phospholipid retention and protein transmission, the porosity of the ceramic membrane is 0.2 μm. Use may alternatively be made of a porosity of 0.1 μm. The high tangential velocity of more than 4 m/s, preferably of 7 m/s, is selected in order to improve protein transmission. Operating temperatures of 30° C. and 50° C. and different transmembrane pressures between 0.4 and 0.6 bar are tested. The batch concentration is carried out as follows: operation takes place firstly at a volume concentration of the buttermilk of between 1.2 and 4, preferably 2, and then instantaneous diafiltration is carried out by addition of water to a level of 50% of the initial volume of buttermilk, and concentration is continued until the volume concentration factor is between 2.5 and 5, preferably 3.

There are multiple applications for the concentrate obtained by the method of the invention. Firstly, the concentrate may be used as an ingredient in liquid form, concentrated or pulverulent for nutritional purposes. Examples include infant, senior and clinical nutrition, or else targeted adult nutrition, for pregnant women and sports persons, for example. In the context of infant formulas, the concentrate constitutes an ingredient and so enables the manufacture of an infant milk comprising a concentrate of this kind.

The concentrate may have other applications, bearing in mind its emulsifying properties. Nonlimiting applications include applications for the preparation of ice-creams, fine bakery products (biscuits, cakes and pastries), chocolates and confectionary, or else cheese spreads.

FIG. 1 illustrates the protein retention rates obtained in tests carried out with (references 2) and without (references 1) prior demineralization, and with the UTP system as described above at 50° C., for varying transmembrane pressures. A high efficacy of the method of the invention, and more particularly of the demineralization effect, is observed on the protein retention rate, which goes from approximately 90% without demineralization to less than 50% with demineralization. Also observed is a substantial influence of the transmembrane pressure on the protein transmission rate when prior demineralization is carried out, this being the reason why a transmembrane pressure of 0.6 bar is preferred.

For the tests carried out with a PG membrane, the buttermilk is also prepared by 8% by mass of solids, from buttermilk powder. The porosity of the membrane is 0.1 μm, the tangential velocity is 5.5 m/s, the operating temperature is 50° C., and the transmembrane pressure is 1 bar. Concentration is carried out as follows: operation takes place initially at a volume concentration of the buttermilk of between 1.2 and 3, preferably 1.5, and then continuous diafiltration is carried out with a flow rate identical to the permeate flow rate and with an amount of water of 100% by volume of initial buttermilk, and concentration is continued until the concentration factor is between 2.5 and 5, preferably 4. The operating parameters tested are indicated in Table 1a below.

For the tests carried with either the UTP system or the PG membrane, diafiltration —a technique known for enhancing the concentration of a product by filtration—is very preferably employed in order to obtain the desired level of phospholipids. The diafiltration may be continuous or instantaneous for the two types of membranes. Preference is given to applying the diafiltration at a low concentration factor, preferably less than 2, in order to best ensure the transmission of the proteins through the membrane. An alternative possibility would be to operate the diafiltration from the start of the concentration step.

With regard to the porosity of the membrane, a porosity of 0.8 μm does not allow sufficient discrimination between the proteins and the phospholipids, and therefore results in an inadequate concentration of phospholipids. The selected porosity for the membrane is therefore less than 0.8 μm, and preferably less than 0.5 μm, in order to ensure an optimal efficacy of yield.

The tangential velocity in the microfiltration operated with the UTP system and the PG membrane is preferably high, being greater than 4 m/s, in order to enhance the transmission of the proteins through the membrane. The temperature of 50° C. is preferably selected in order to increase the flow rate of permeate, without altering the separation capacities of the method.

Reference is made to Table 1 b, which presents the results of tests conducted on an organic membrane, on a PG membrane, with the UTP system, according to the operating parameters defined above. For all of the tests, the volume concentration factor applied is 3. The underlined entries correspond to tests in which the method parameters are outside the invention, and the non-underlined entries correspond, conversely, to tests in which the method parameters are within the scope of the invention. The concentration factor (CF) indicated in Table 1a reports the protein and phospholipid retention rates in isolation from the variability in the solids content of the buttermilk intended for concentration.

The phospholipid concentrate obtained by the method of the invention has a ratio between its phospholipid solids content and its protein solids contents of more than 6.5. This ratio is advantageously more than 7.

TABLE 1a
Operating parameters of the tests conducted on organic
membrane, on PG membrane, and with the UTP system

| | | | | Cut-off | | | |
|---|---|---|---|---|---|---|---|
| | | With or without | | limit | | | |
| Test | Membrane type | demineralization | Buttermilk | (μm) | T | Diafiltration | TMP |
| 1 | Organic | Without | Reconstituted | 0.1 | 50° C. | Continuous | 1 bar |
| 2 | membrane | With | Reconstituted | 0.1 | 50° C. | Continuous | 1 bar |
| 3 | PG membrane | Without | Reconstituted | 0.1 | 50° C. | Continuous | 1 bar |
| 4 | | With | Reconstituted | 0.1 | 50° C. | Continuous | 1 bar |
| 5 | | With | Reconstituted | 0.1 | 50° C. | Continuous | 1 bar |
| 6 | | With | Fresh | 0.1 | 50° C. | Continuous | 1 bar |
| 7 | | With | Fresh | 0.1 | 50° C. | Continuous | 1 bar |
| 8 | | Without | Reconstituted | 0.2 | 50° C. | Instantaneous | 0.6 bar |
| 9 | | Without | Fresh | 0.2 | 50° C. | Instantaneous | 0.6 bar |
| 10 | | With | Reconstituted | 0.2 | 50° C. | Instantaneous | 0.6 bar |
| 11 | | With | Fresh | 0.2 | 50° C. | Instantaneous | 0.6 bar |
| 12 | | Without | Reconstituted | 0.2 | 30° C. | Instantaneous | 0.6 bar |
| 13 | | With | Reconstituted | 0.2 | 30° C. | Instantaneous | 0.6 bar |
| 14 | UTP | With | Reconstituted | 0.2 | 30° C. | Instantaneous | 0.6 bar |
| 15 | system | Without | Reconstituted | 0.2 | 50° C. | Instantaneous | 0.4 bar |
| 16 | | With | Reconstituted | 0.2 | 50° C. | Instantaneous | 0.4 bar |
| 17 | | With | Reconstituted | 0.2 | 50° C. | Instantaneous | 0.4 bar |
| 18 | | Without | Reconstituted | 0.2 | 50° C. | Instantaneous | 0.6 bar |
| 19 | | With | Reconstituted | 0.2 | 50° C. | Instantaneous | 0.6 bar |
| 20 | | With | Reconstituted | 0.2 | 50° C. | Instantaneous | 0.6 bar |
| 21 | | With | Fresh | 0.2 | 50° C. | Instantaneous | 0.6 bar |

TABLE 1b
Comparative results of the tests conducted on organic membrane, on PG membrane, and with the UTP system

| | | | | | | Protein | Phospholipid | Protein | Phospholipids |
|---|---|---|---|---|---|---|---|---|---|
| | Solids | Protein | Phospholipid | | | CF**/ | content | content | (% SC)/Proteins |
| | retention * | retention * | retention * | Protein | Phospholipid | Phospholipid | (% SC) of | (% SC) of | (% SC) in the |
| Test | (%) | (%) | (%) | CF** | CF** | CF | concentrate | concentrate | concentrate |
| 1 | 41 | 86 | 90 | 2.2 | 2.2 | 1.0 | 4.2 | 69 | 6.0% |
| 2 | 38 | 84 | 87 | 2.1 | 2.3 | 0.9 | 4.3 | 73 | 5.9% |
| 3 | 41 | 85 | 74 | 2.1 | 1.8 | 1.2 | 3.3 | 74 | 4.5% |
| 4 | 32 | 67 | 83 | 2.1 | 2.6 | 0.8 | 4.8 | 71 | 6.7% |
| 5 | 29 | 57 | 87 | 2 | 3.0 | 0.7 | 5.6 | 72 | 7.8% |
| 6 | 37 | 66 | 75 | 1.8 | 2.0 | 0.9 | 4.5 | 63 | 7.2% |
| 7 | 20 | 38 | 80 | 1.9 | 3.9 | 0.5 | 8 | 67 | 11.9% |
| 8 | 44 | 88 | ND | 2 | ND | ND | ND | 64 | ND |
| 9 | 46 | 89 | ND | 2 | ND | ND | ND | 69 | ND |
| 10 | 26 | 43 | 67 | 1.7 | 2.6 | 0.7 | 4.8 | 55 | 8.7% |
| 11 | 27 | 38 | 71 | 1.4 | 2.6 | 0.5 | 5.3 | 49 | 10.9% |
| 12 | 39 | 78 | 74 | 2 | 1.9 | 1.1 | 3.5 | 65 | 5.4% |
| 13 | 31 | 46 | 76 | 1.5 | 2.5 | 0.6 | 4.7 | 52 | 9.0% |
| 14 | 28 | 45 | 74 | 1.6 | 2.6 | 0.6 | 4.8 | 53 | 9.1% |
| 15 | 42 | 88 | 97 | 2.1 | 2.3 | 0.9 | 4.5 | 73 | 6.2% |
| 16 | 29 | 43 | 101 | 1.5 | 3.6 | 0.4 | 5.9 | 54 | 10.9% |
| 17 | 27 | 40 | 68 | 1.5 | 2.6 | 0.6 | 4.7 | 50 | 9.4% |
| 18 | 44 | 88 | 102 | 2 | 2.3 | 0.9 | 3.8 | 64 | 5.9% |
| 19 | 21 | 37 | 81 | 1.8 | 3.3 | 0.5 | 6.2 | 56 | 11.1% |
| 20 | 25 | 43 | 67 | 1.7 | 2.6 | 0.7 | 4.8 | 55 | 8.7% |
| 21 | 33 | 38 | 71 | 1.4 | 2.6 | 0.5 | 5.3 | 48.9 | 10.8% |

*Retention rate: ratio between the mass in the retentate and the mass in the buttermilk before microfiltration;
**CF—Concentration factor: ratio between the mass fraction of solids in the retentate and the mass fraction of solids in the buttermilk before microfiltration

Firstly it is observed that when the microfiltration is conducted on an organic membrane (tests 1 and 2), the protein retention rate remains high, at more than 80%, even when the buttermilk has been depleted of calcium beforehand. Contrary to expectation, therefore, the demineralization has no effect on the transmission of the proteins. There also remains a difficulty with cleaning of this type of membrane after the concentration of the buttermilk.

As for the tests carried out on a PG membrane, it is observed that the protein retention rate is significantly lowered when the buttermilk is depleted of calcium beforehand. Although the protein concentration factor is not significantly lowered, the phospholipid concentration factor is greatly increased, thereby resulting on the one hand in a ratio between the protein concentration factor and the phospholipid concentration factor that is lower relative to that obtained without demineralization, and in a ratio between phospholipids and proteins in the concentrate that is much higher than that obtained without demineralization. It is also observed that the results are comparable between the reconstituted buttermilk and the fresh buttermilk.

With regard to the tests carried out with the UTP system, it is also observed that the protein retention rate is significantly lowered when the buttermilk has been depleted of calcium beforehand, for a high phospholipid retention rate. The protein concentrator factor is lower relative to tests conducted without demineralization, thereby resulting in a substantial drop in the ratio between the protein concentration factor and the phospholipid concentration factor. The ratio between phospholipids and proteins in the concentrate, moreover, is very much greater than that obtained without demineralization. As for the tests carried out with the PG membrane, it is observed that the results are comparable between the reconstituted buttermilk and the fresh buttermilk.

On the basis of these results, two joint parameters are defined that are characteristic of the method of the invention. These are the ratio between the protein concentration factor and the phospholipid concentration factor, which is less than 1, and the protein retention rate, which is less than 70%. Preferably, in order to ensure a better phospholipid concentration in the concentrate, the ratio between the protein concentration factor and the phospholipid concentration factor is less than 0.9.

Described below are two nonlimiting working examples (Examples 1 and 2) of the invention, and an example characterizing the lipid compounds and protein compounds in the concentrate of the invention and in a concentrate produced without prior demineralization (Example 3).

### Example 1

Resin Preparation:

The resin used is a weak cationic-ion exchange resin, consisting of carboxyl groups on an acrylic matrix (IMAC HP336). The resin is originally in hydrogen H+ form. Regeneration with a sodium hydroxide NaOH and potassium hydroxide KOH solution is applied to convert 20 liters of virgin resin into a sodium and potassium form. For this, an equimolar solution of 75 L is prepared at 0.5 mol/L of NaOH and 0.5 mol/L of KOH. The solution is eluted on the bed of resin at a flow rate of 6 BV (resin bed volume)/h. The resin is subsequently rinsed with water at the same flow rate until the conductivity of the water exiting the bed is less than 1 mS/cm.

Passage of Buttermilk Over the Resin:

Milk buttermilk obtained from butter production was subjected to preliminary treatment by bactofuging and pasteurizing. It has the composition given in the table below. A volume of 40 BV of buttermilk is eluted on the resin at a volume flow rate of 10 BV/h. The product is then driven out with 1 BV of water.

TABLE 1
Composition of buttermilk before and after
treatment on the ion exchange column

| | | |
|---|---|---|
| | Before ion | After ion |
| Buttermilk composition | exchange | exchange |

| | | | |
|---|---|---|---|
| Solids content | % | 7.8 | 7.8 |

Composition by mass of solids

| | | | |
|---|---|---|---|
| Proteins (=N × 6.38) | % | 35.9 | 35.3 |
| Na | mg/100 g | 477 | 1202 |
| K | mg/100 g | 1711 | 3019 |
| Ca | mg/100 g | 1102 | 4 |
| Mg | mg/100 g | 119 | 1 |
| Ash | % | 8.0 | 9.5 |

The resin is then regenerated with a volume of 46 L of hydrochloric acid with a concentration of 1.7 mol/L. The percolation rate of the solution applied is 6 BV/h. The resin is then rinsed with water until the conductivity at the column exit is 1 mS/cm. The resin is subsequently regenerated in Na/K form using the same preparation protocol as before.

Concentration by Microfiltration:

The buttermilk treated on the ion exchange column is heated to 50° C. and then concentrated via microfiltration. The microfiltration system used is a module with 19 Membralox GP EP1940 membranes with a cut-off limit of 0.1 μm. The buttermilk is concentrated batchwise, i.e., with recirculation of the retentate in a tank until the desired volume concentration factor is attained. When the volume concentration factor is 1.5, a continuous diafiltration is applied. For this purpose, water is added to the tank at a continuous flow rate of 160 L/h; the total volume of water added is 80% of the initial volume of buttermilk. The diafiltration water is likewise at 50° C. and the temperature in the method is maintained at 50° C. by means of a heat exchanger. The filtration is controlled at a constant transmembrane pressure of 1 bar for the entire concentration. The permeate is recovered in a tank. Concentration is at an end when the volume concentration factor VCF is 4, relative to the initial volume of buttermilk.

The composition of the buttermilk at the start and end of concentration is indicated in the table below.

TABLE 2
Composition of buttermilk before concentration and
after depletion in calcium, and after concentration

| | | |
|---|---|---|
| Buttermilk composition | Initial | Concentrated |

| | | | |
|---|---|---|---|
| Solids content | % | 7.8 | 6.7 |
| Proteins | % SC | 35.3 | 67.3 |
| Phospholipids | % SC | 2.0 | 8.0 |

### Example 2

After a first ion exchange step exactly the same as that described in example 1, the calcium-depleted buttermilk undergoes batchwise concentration on a 0.2 μm ceramic microfiltration membrane. The module is made up of seven Membralox P3730 membranes. The method is equipped with a UTP (Uniform Transmembrane Pressure) system, meaning that it is composed of a permeate-side recirculation pump. The recirculation pump operates at a flow rate of 42 m³/h so as to reach a velocity of 7 m/s in the filtration module. Concentration is performed at a transmembrane pressure which is kept constant at 0.6 bar for the entire step of the method. As for example 1, the buttermilk is first heated to 50° C. and maintained at this temperature by means of a heat exchanger.

The buttermilk is concentrated to a VCF of 2, and then an instantaneous diafiltration is applied, by adding a volume of water equal to the volume of concentrated product in the tank. Concentration is then continued to a VCF of 3.

The compositions of the initial buttermilk and of the concentrated buttermilk are given in the table below.

TABLE 3
Composition of the buttermilk before and after concentration

| | | |
|---|---|---|
| Buttermilk composition | Initial | Concentrated |

| | | | |
|---|---|---|---|
| Solids content | % | 7.8 | 6.7 |
| Proteins | % ES | 35.3 | 67.3 |
| Phospholipids | % ES | 2.0 | 8.0 |

### Example 3

Characterization tests were conducted on the liquid phospholipid concentrate of the invention as obtained in Example 1, and on a liquid concentrate prepared in accordance with Example 1 but without a demineralization step in the production method.

The first test conducted involves performing centrifugation at 31500 rpm on the two concentrates. According to the literature, this centrifugation speed corresponds to the speed which ensures precipitation of the casein micelles.

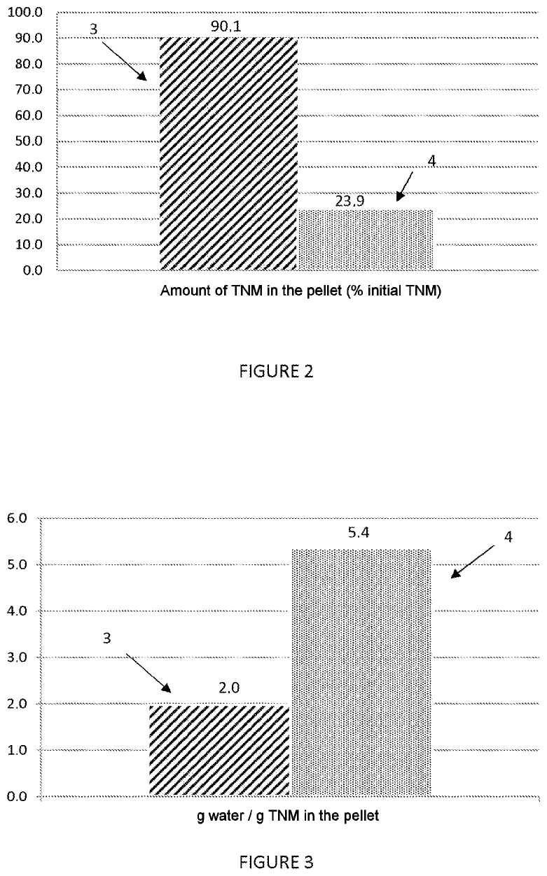

FIG. 2 illustrates the amount of Total Nitrogenous Matter (MAT) found in the centrifugation pellet, relative to the initial percentage of Total Nitrogenous Matter, for—respectively—the concentrate obtained without demineralization (reference 3) and the concentrate of the invention (reference 4). Substantially less nitrogenous matter is observed in the concentrate of the invention. The identification of the casein micelles in the concentrate obtained without demineralization (and their virtual absence from the concentrate of the invention) is supported by the results presented in FIG. 3. FIG. 3 illustrates the weight, expressed in grams, of water relative to the weight, expressed in grams, of Total Nitrogenous Matter (TNM) in the centrifugation pellet for—respectively—the concentrate obtained without demineralization (reference 3), and the concentrate of the invention (reference 4). For the concentrate obtained without demineralization, the water retention value of 2 grams of water per gram of Total Nitrogenous Matter that is obtained is in agreement with that known from the literature for casein micelles. The higher value, of 5.4 grams of water per gram of Total Nitrogenous Matter for the concentrate of the invention, indicates that the total nitrogenous matter present in this concentrate corresponds to the presence of micelle fragments, and not of casein micelles. The concentrate of the invention therefore contains no casein micelles, in contrast to the concentrate obtained without demineralization.

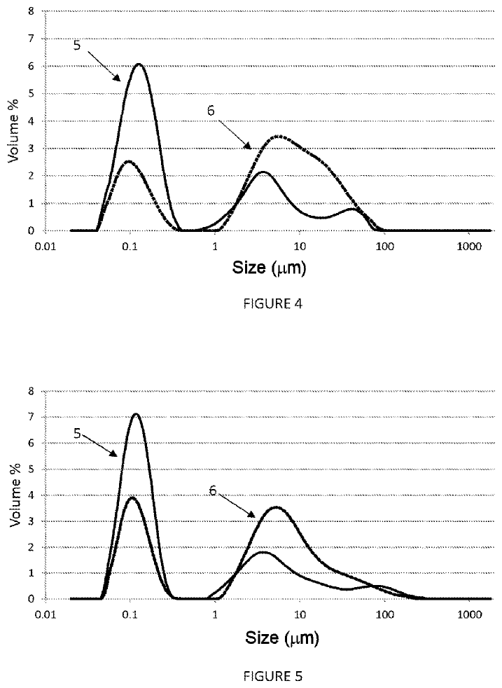

Laser particle sizing measurements are carried out on the liquid concentrate of the invention (reference 6) and on the concentrate obtained without demineralization (reference 5). FIG. 4 illustrates the results obtained when EDTA (ethylenediaminetetraacetic acid) is added to the concentrates, and FIG. 5 illustrates the results when EDTA and SDS (sodium dodecyl sulfate) are added to the concentrates. EDTA is a calcium chelator and causes disaggregation of the casein micelles. SDS disaggregates the fatty globules from one another.

In the two FIGS. 4 and 5, a markedly greater presence of the caseins is observed (respectively areas under the curves corresponding to the peaks centered on 0.1 μm) for the concentrate obtained without demineralization, again demonstrating the substantial presence of casein micelles when demineralization on an ion exchange column is not applied.

Also observed, in the presence of SDS, for the concentrate of the invention, is a reduction in the area under the curve positioned between 10 and 100 μm, and a concomitant increase in the area under the curve positioned between 1 and 10 μm, whereas, for the concentrate obtained without demineralization, these areas show virtually no change in the presence of SDS. These results demonstrate the presence, in the concentrate of the invention, of agglomerations of membranes of fatty globules (with a size of between 10 and 100 μm), which undergo disaggregation in the presence of SDS. The presence of membranes of fatty globules indicates the presence of phospholipids, and so these results indicate and support the substantial concentration in phospholipids that is obtained by virtue of the method of the invention.

1. A method for producing a concentrate of phospholipids from a liquid dairy composition comprising at least caseins and phospholipids, wherein the method comprises:
passing the dairy composition over a cationic resin in an ion exchange column and obtaining a calcium-depleted dairy composition,
concentrating the phospholipids of the calcium-depleted dairy composition by controlled transmembrane pressure gradient microfiltration and obtaining a microfiltration retentate, and
recovering the microfiltration retentate and obtaining a phospholipid concentrate.

2. The method as claimed in claim 1, wherein the dairy composition comprises at least one selected from the group consisting of milk buttermilk and concentrated buttermilk.

3. The method as claimed in claim 1,
wherein a ratio between a protein concentration factor and a phospholipid concentration factor is less than 1, each of the concentration factors corresponding to a ratio between the mass fraction of solids in the microfiltration retentate and a mass fraction of solids in the buttermilk before microfiltration, and
wherein a protein retention rate induced by the microfiltration is less than 70%.

4. The method as claimed in claim 3, wherein the ratio between the protein concentration factor and the phospholipid concentration factor is less than 0.9.

5. The method as claimed in claim 1, wherein the cationic resin of the ion exchange column is a weak cationic resin.

6. The method as claimed in claim 1, wherein a diafiltration is carried out during the phospholipid concentration.

7. The method as claimed in claim 6, wherein the diafiltration is applied for a volume concentration factor of less than 3.

8. The method as claimed in claim 1, wherein the phospholipid concentration is carried out at 50° C.

9. The method as claimed in claim 1, wherein a tangential velocity applied to the microfiltration is greater than 4 m/s.

10. The method as claimed in claim 1, wherein a mean pore size of the microfiltration membrane is less than 0.8 micrometer.

11. The method as claimed in claim 6, wherein the mean pore size of the microfiltration membrane is less than 0.5 micrometer.

12. The method as claimed in claim 11, wherein a transmembrane pressure is in a range of from 0.4 bar to 2 bar.

13. The method as claimed in claim 12, wherein the transmembrane pressure is in a range of from 0.6 bar to 1 bar.

14. The method as claimed in claim 1, wherein the phospholipid liquid concentration is carried out on a permeability gradient membrane.

15. The method as claimed in claim 14, wherein the permeability gradient membrane has a mean pore size of 0.1 micrometer.

16. The method as claimed in claim 14, wherein a transmembrane pressure is maintained at 1 bar.

17. The method as claimed in claim 15, wherein a continuous diafiltration is applied when the volume concentration factor is a range of from 1.2 to 3.

18. The method as claimed in claim 15, wherein the concentration is carried out until a volume concentration factor of the dairy composition is in a range of from 2.5 to 5.

19. The method as claimed in claim 1, wherein the phospholipid concentration is carried out on a ceramic membrane coupled to a system for harmonizing a transmembrane pressure.

20. The method as claimed in claim 19, wherein a mean pore size of the ceramic membrane is in a range of from 0.1 to 0.2 micrometer.

21. The method as claimed in claim 19, wherein the transmembrane pressure is maintained at 0.6 bar.

22. The method as claimed in claim 19, wherein a continuous diafiltration is applied when a volume concentration factor is in a range of from 1.2 to 4.

23. The method as claimed in claim 19, wherein the concentration is carried out until a volume concentration factor of the dairy composition is in a range of from 2.5 to 5.
